(19) United States
(12) Patent Application Publication
(10) Pub. No.: US 2026/0130887 A1
(43) Pub. Date: May 14, 2026

(54) ORAL COMPOSITIONS COMPRISING LP(A) INHIBITOR COMPOUNDS

(71) Applicant: ELI LILLY AND COMPANY, Indianapolis, IN (US)

(72) Inventors: Sydney Marie BUTIKOFER, Indianapolis, IN (US); Dale Eldon GREENWOOD, Fishers, IN (US); Faiz Nisar KHAN, Indianapolis, IN (US); Jennifer Anne MCMAHON, Zionsville, IN (US); Mellie June PAULINES, Indianapolis, IN (US); Sofiane SAOUANE, Indianapolis, IN (US); Jon Gordon SELBO, Noblesville, IN (US); Christine Lynn SKAGGS, Camby, IN (US)

(21) Appl. No.: 19/388,561

(22) Filed: Nov. 13, 2025

Related U.S. Application Data
(60) Provisional application No. 63/720,478, filed on Nov. 14, 2024.

(51) Int. Cl. A61K 31/4025; A61K 9/00; A61K 9/20; C07D 207/09

(52) U.S. Cl. CPC A61K 31/4025; A61K 9/0053; A61K 9/2013; A61K 9/2054; C07D 207/09

(57) ABSTRACT

Oral compositions including a Lp(a) inhibitor compound and histidine. Oral compositions including muvalaplin and histidine. New polymorphs of muvalaplin, such as muvalaplin hydrate Form A, muvalaplin hydrate Form B, muvalaplin hemihydrate, and muvalaplin hydrate 1:6.5 mol eq. Oral compositions comprising muvalaplin hemihydrate and/or muvalaplin hydrate 1:6.5 mol eq. A method of treating a cardiovascular disease, treating an atherosclerotic cardiovascular disease, and/or lowering the level of Lp(a) in an individual by administering one of the oral compositions.

### FIELD OF THE INVENTION

The present invention relates to oral compositions comprising an Lp(a) inhibitor compound, such as muvalaplin, and histidine. The present invention also relates to oral compositions comprising an Lp(a) inhibitor compound, histidine, and reduced levels of nitrosamine, such as less than 50 ppm of nitrosamine. The present invention also relates to new polymorphs of muvalaplin, such as hydrates of muvalaplin. The present invention also relates to oral compositions comprising an Lp(a) inhibitor compound, such as muvalaplin, and an excipient with low nitrite content.

### BACKGROUND OF THE INVENTION

There have been significant advances in treating cardiovascular disease (CVD). Despite treatment advances, patients continue to experience cardiovascular disease events such as angina, myocardial infarction, and stroke, which if untreated, lead to death. Lipid disorder or dyslipidemia remains a major risk factor for CVD. Lipid disorders can be divided into four general risk factors: elevated low-density lipoprotein cholesterol (LDL-c), low high-density lipoprotein cholesterol (HDL-c), elevated triglycerides (TG), and elevated lipoprotein(a) (Lp(a)). There are a variety of treatment regimens targeting elevated LDL-c, low HDL-c, and elevated triglycerides. There are few treatment options for patients with elevated Lp(a) concentrations. In some cases, apheresis may be used to filter the blood to remove LDL and Lp(a); however, the effects are temporary and typically need to be repeated every two weeks. Proprotein convertase subtilisin/kexin type 9 (PCSK9) inhibitors reduce Lp(a) by approximately 25% and niacin produces modest reductions in Lp(a), but neither are approved for Lp(a) reduction. There is no pharmaceutical treatment approved to lower Lp(a) levels. The physiological function of Lp(a) is complex; however, it is reported that elevated Lp(a) plasma level is an independent risk factor for CVD.

Lp(a) may exhibit both prothrombotic and antithrombotic properties, atherogenic and atherothrombotic properties. Lp(a) may inhibit fibrinolysis and accumulate in the vascular wall inducing thrombogenesis and atherosclerotic lesions. Plasma levels of Lp(a) vary substantially among individuals. Unlike the other risk factors, Lp(a) plasma levels do not vary significantly with diet and exercise. Lp(a) plasma levels are primarily determined by genetic predisposition.

Lp(a) resembles LDL-c in that it includes an LDL lipid core with the attendant apolipoprotein B (apoB), but unlike LDL-c, Lp(a) also contains a unique apolipoprotein(a) (apo(a)) bound covalently to the apoB via a disulfide bond. Apo(a) is synthesized in the liver. The assembly of Lp(a) from apo(a) and LDL particles can occur in hepatocytes, on the cell wall or in plasma. Inhibition of the assembly of the LDL particle with apo(a) may reduce Lp(a) levels.

It has been previously reported, such as in U.S. Pat. No. 11,286,249, that certain pyrrolidine compounds, such as (2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid or muvalaplin, can be used to reduce Lp(a) levels.

However, certain secondary amines, such as the nitrogen atom in an unsubstituted pyrrolidine compound, can react under certain conditions to form nitrosamine functional groups. N-nitrosamines (commonly called nitrosamines) are considered probable or possible carcinogens according to the International Agency for Research on Cancer, an agency within the World Health Organization. Thus, there is a need for an oral composition comprising a Lp(a) inhibitor compound with a reduced level of nitrosamine, such as less than 50 ppm without impacting other pharmaceutically relevant oral composition properties such as disintegration time or the overall stability of the composition.

Additionally, while Lp(a) inhibitor compounds, such as muvalaplin, are previously reported in U.S. Pat. No. 11,286,249, there is a need for alternative solid-state forms of Lp(a) inhibitor compounds, such as muvalaplin, with improved stability.

### SUMMARY OF THE INVENTION

Accordingly, the present invention is directed to oral compositions comprising an Lp(a) inhibitor compound and histidine. It has been unexpectedly found that the addition of histidine to the oral composition can lead to oral compositions with reduced level of nitrosamine without significantly impacting the disintegration time or overall stability of the oral composition.

Additionally, the present invention is directed to oral compositions comprising an Lp(a) inhibitor compound, histidine, and a bulking agent, such as microcrystalline cellulose, with a low amount of nitrite. It has been unexpectedly found that the addition of a bulking agent with a low amount of nitrite to the oral composition can lead to oral compositions with reduced levels of nitrosamine.

In an aspect, provided herein is an oral composition comprising: (a) an Lp(a) inhibitor compound of the formula:

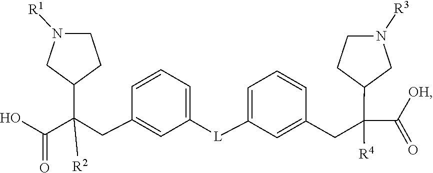

wherein:
L is selected from the group consisting of —CH₂NHCH₂—, —CH₂NH—, —NH—, —S—, —S(O)—, —S(O)₂—, —O—, —OCH₂—, —OCH₂CH₂O—, —NHSO₂NH—,

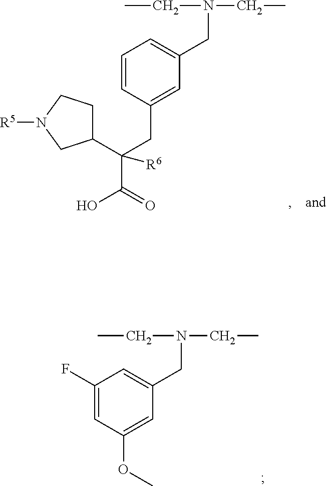

R¹, R², R³, R⁴, R⁵, and R⁶ are each independently selected from the group consisting of H and CH₃; or a pharmaceutically acceptable salt thereof; and (b) histidine.

In another aspect, provided herein is an oral composition comprising: (a) an Lp(a) inhibitor compound of the formula:

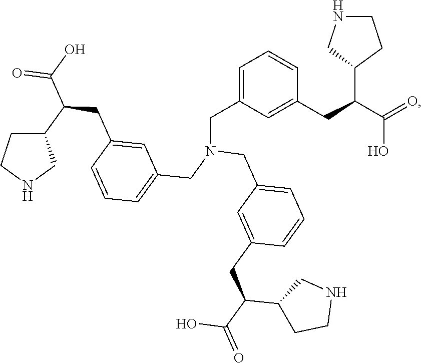 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

or a pharmaceutically acceptable salt thereof; and (b) histidine.

In a further aspect, provided herein is an oral composition comprising: (a) an Lp(a) inhibitor compound of the formula:

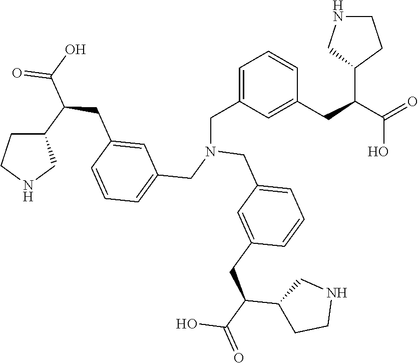 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

or a pharmaceutically acceptable salt thereof; and (b) from about 1.0% to about 3.0%, by weight of the oral composition, of histidine, wherein the oral composition is a tablet and comprises less than 50 ppm of nitrosamine.

In a further aspect, provided herein is any one of the disclosed oral compositions for use in treating a cardiovascular disease in an individual.

In a further aspect, provided herein is a method of treating an individual with elevated Lp(a) levels, the method comprising: orally administering to the individual any one of the disclosed oral compositions.

In a further aspect, provided herein is a method of treating atherosclerotic cardiovascular disease in an individual, the method comprising: orally administering to the individual any one of the disclosed oral compositions.

The present invention is also directed to novel polymorphs of muvalaplin, such as muvalaplin hydrate (Form A) and muvalaplin hydrate (Form B).

In an aspect, provided herein is a compound of the formula:

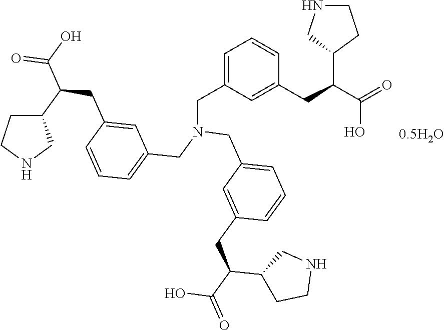 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

In a further aspect, provided herein is a compound of the formula:

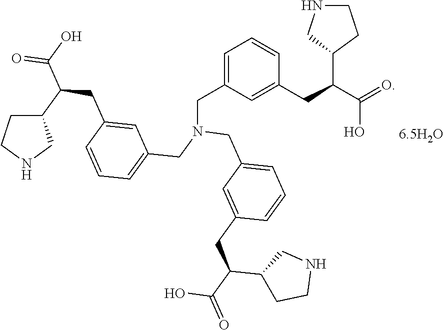 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

In a further aspect, provided herein are compositions comprising a hydrate of muvalaplin.

### BRIEF DESCRIPTION OF THE FIGURES

FIG. 1 shows the XPRD pattern of Example 2.

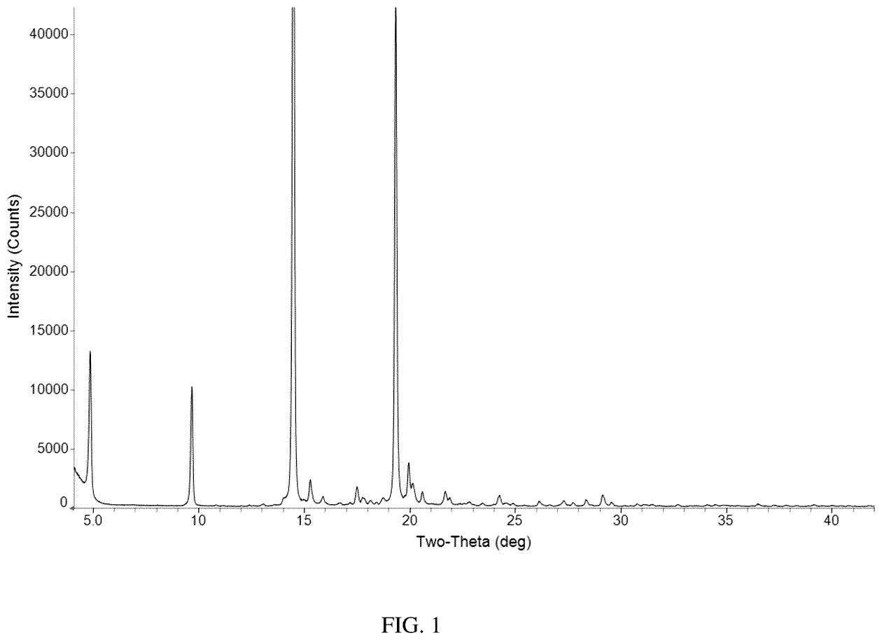

FIG. 2 shows the SCXRD of Example 2.

FIG. 3 shows the XPRD pattern of Example 3.

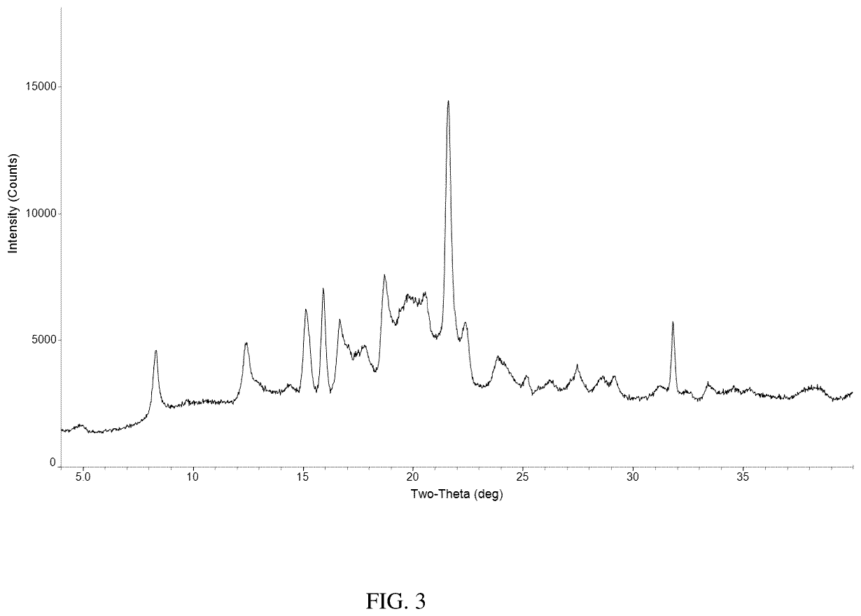

FIG. 4 shows the SXRD of Example 3.

### DETAILED DESCRIPTION OF THE INVENTION

The section headers below are provided for convenience only. In some cases, a compound can fall within one or more section headers listed below. For example, microcrystalline cellulose can be considered a bulking agent and/or a binding agent.

### Oral Compositions

The present invention relates to oral compositions comprising an Lp(a) inhibitor compound and histidine. Additionally, the oral composition can comprise other optional ingredients, as described below.

The provided oral compositions include minimal levels of nitrosamine. As is known to a person of ordinary skill in the art, compounds comprising secondary amines, such as those including pyrrolidine functional groups, can result in the formation of nitrosamine functional groups in the presence of nitrous acid (HONO). Nitrous acid can be present in (1) aqueous solutions and (2) under acidic conditions in the presence of nitrite salts, which can be found in many excipients known in the art to be suitable for pharmaceutical compositions. It has been unexpectedly found that the addition of histidine to the oral composition can lead to the minimization and/or the complete prevention of the formation of nitrosamine byproducts after the manufacture of the oral composition.

Additionally, it has been unexpectedly found that by minimizing the amount of nitrate salts present in the oral composition that the levels of nitrosamine byproduct can be further reduced. For example, if the other excipients are selected for low nitrite content, the amount of nitrosamine formation can be further reduced.

Suitable oral compositions include oral compositions with a reduced levels of nitrosamine. Reduced levels of nitrosamine can include less than 50 ppm, less than 25 ppm, less than 15 ppm, less than 12.5 ppm, from about 1 ppb to about 50 ppm, from about 1 ppb to about 25 ppm, from about 1 ppb to about 15 ppm, from about 1 ppb to about 12.5 ppm, from about 0.5 ppm to about 50 ppm, from about 0.5 ppm to about 25 ppm, from about 0.5 ppm to about 15 ppm, or from about 0.5 ppm to about 12.5 ppm. Other reduced levels of nitrosamine include the oral composition being substantially free of, essentially free of, or free of nitrosamine.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels, such as 1 ppb or 0.5 ppm for nitrosamine. It is meant to include compositions whereby the indicated material is present only as an impurity of one of the other materials deliberately added.

The oral composition can be a tablet composition or a capsule composition.

Accordingly, disclosed herein are tablet compositions comprising any one of the Lp(a) inhibitor compounds disclosed herein, histidine, and optionally one or more pharmaceutically acceptable excipients, such as a bulking agent, disintegrant, binding agent, and/or a lubricant.

Tablets can be made by high shear wet granulation. The high shear wet granulation manufacturing process may be performed by blending the powders by diffusion, convection or pneumatic mixing. The powders may be granulated by adding water, water plus nitrosamine inhibitor, water plus binder or water plus binder and nitrosamine inhibitor to form the granulation. The milling steps can be performed by impact, cutting, compression, screening mills or separators. After granulation, the powders may be dried by direct heating—static solids bed, direct heating—moving solids bed, direct heating—fluidized bed, indirect conduction heating—moving solids bed, or indirect conduction heating—static solids bed. The tablets may be compressed by gravity, power assisted or centrifugal tableting machines. The core tablet may be film coated using non-perforated or perforated coating systems. All processes may be performed as a batch or a continuous process.

Also disclosed herein is a capsule composition comprising any one of the Lp(a) inhibitor compounds and histidine. The capsule composition can include a suitable capsule, such as gelatin or hypromellose with or without a gelling agent. The capsule can be a size 000, 00, 0, 1, or 2 depending on the total amount of material added.

Lp(a) inhibitor compound

The oral composition of the present invention comprises a Lp(a) inhibitor compound. The Lp(a) inhibitor compound can be used in combination with the other oral composition compounds, as described herein.

Suitable Lp(a) inhibitor compounds can comprise a compound of Formula I, or a pharmaceutically acceptable salt thereof, wherein L is selected from the group consisting of —CH₂NHCH₂—, —CH₂NH—, —NH—, —S—, —S(O)—, —S(O)₂—, —O—, —OCH₂—, —OCH₂CH₂O—, —NHSO₂NH—,

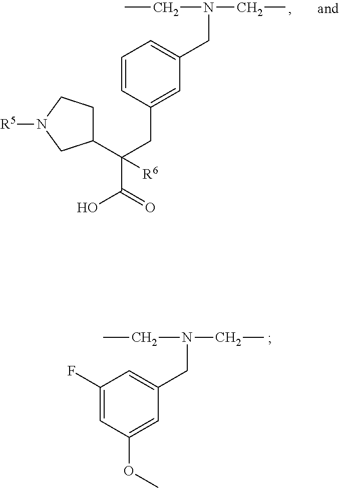

R¹, R², R³, R⁴, R⁵, and R⁶ are each independently selected from the group consisting of H and CH₃

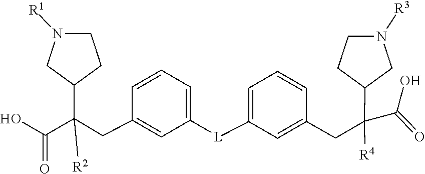

Formula I. Lp(a) Inhibitor Compound

In an aspect, one of R₁, R₃, or Rs, if present, is H. In another aspect, each of R₁, R₃, and R₅, if present, is H.

The Lp(a) inhibitor compound can also comprise a compound of Formula II, or a pharmaceutically acceptable salt thereof.

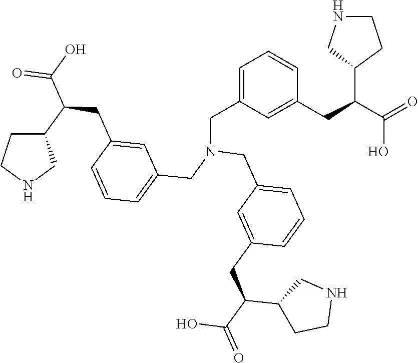 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

Formula II. Lp(a) Inhibitor Compound, (2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid, muvalaplin

The Lp(a) inhibitor compound can also comprise a compound of Formula III, IV, or V.

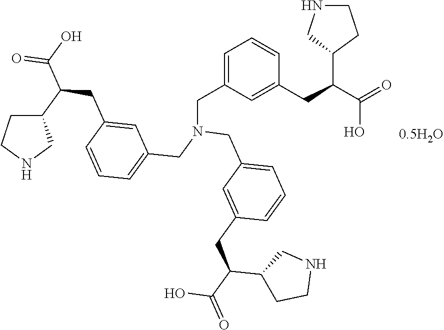 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

Formula III. Lp(a) Inhibitor Compound, (2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid hemihydrate, muvalaplin hemihydrate

The present invention also discloses new polymorphs of Muvalaplin, such as muvalaplin hydrate (Form A) and muvalaplin hydrate (Form B).

Muvalaplin hydrate (Form A) can be characterized by an XRPD pattern using CuKα radiation as having diffraction peaks (20°) as described in Table 1 below. In an aspect, Muvalaplin hydrate (Form A) can be characterized by an XRPD pattern using CuKα radiation as having diffraction peaks (2θ) at 14.3°±0.2° 2θ and one or more of the peaks selected from the group consisting of 4.7° and 9.5°±0.2° 2θ. In another aspect, Muvalaplin hydrate (Form A) can be characterized by an XRPD pattern using CuKα radiation as having diffraction peaks (2θ) at 4.7°, 9.5°, and 14.3°±0.2° 2θ. In another aspect, Example 2 can be characterized by an XRPD pattern using CuKα radiation as having diffraction peaks (2θ) at 4.7°, 9.5°, 14.3°, and 19.2°±0.2° 2θ. In another aspect, Muvalaplin hydrate (Form A) can be characterized by an XRPD pattern using CuKα radiation as having diffraction peaks (2θ) of 4.7°, 9.5°, 14.3°, 15.2°, 17.4°, and 19.2°±0.2° 2θ.

Analysis of a single crystal of Muvalaplin hydrate (Form A) shows the material to be a hemihydrate that crystallizes in the monoclinic space group P21. Muvalaplin hydrate (Form A) can be characterized by having unit cell parameters at 100 Kelvin of about a=11.25 Å, b=18.21 Å, c=18.65 Å, α=90°, β=100.19°, and γ=90°.

Muvalaplin hydrate (Form B) can be characterized by an XRPD pattern using CuKα radiation as having diffraction peaks (2θ) as described in Table 3 below. In an aspect, Muvalaplin hydrate (Form B) can also be characterized by an XRPD pattern using CuKα radiation as having diffraction peaks (2θ) at 12.3°±0.2° 2θ in combination with one or more of the peaks selected from the group consisting of 8.2° and 20.6°±0.2° 2θ°. In another aspect, Muvalaplin hydrate (Form B) can also be characterized by an XRPD pattern using CuKα radiation as having diffraction peaks (2θ) at 8.2°, 12.3°, and 20.6°±0.2° 2θ. In another aspect, Muvalaplin hydrate (Form B) can also be characterized by an XRPD pattern using CuKα radiation as having diffraction peaks (2θ) at 8.2°, 12.3°, 16.4°, 20.6°, and 21.1°±0.2° 2θ°.

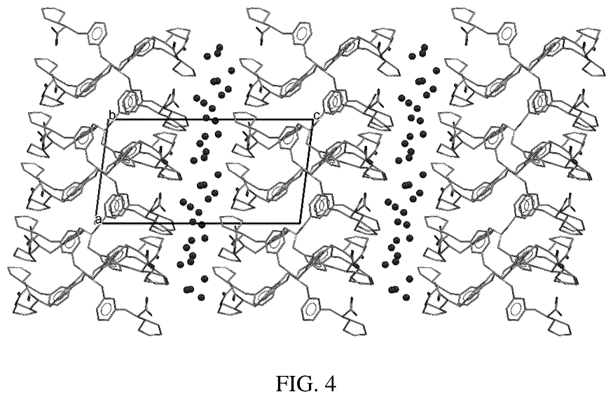

Analysis of a single crystal of Muvalaplin hydrate (Form B) shows the material to be a 6.5 mol equivalent hydrate that crystallizes in the monoclinic space group P21. Table 4 outlines the unit cell parameters and properties of a single crystal isolated from Muvalaplin hydrate (Form B). FIG. 4 shows the bonding scheme (hydrogen atoms omitted for clarity). A single crystal isolated from Muvalaplin hydrate (Form B) can be characterized by having unit cell parameters at 100 Kelvin of about a=11.06 Å, b=18.52 Å, c=21.58 Å, α=90°, β=97.36°, and γ=90°.

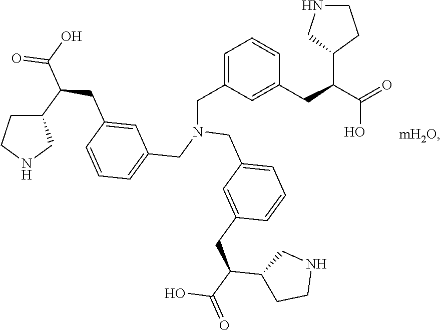 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

Formula IV. Lp(a) Inhibitor Compound, (2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid hydrate (1 mol of Lp(a) inhibitor compound to 6.5 mol of water), muvalaplin hydrate (1 mol of muvalaplin to 6.5 mol of water)

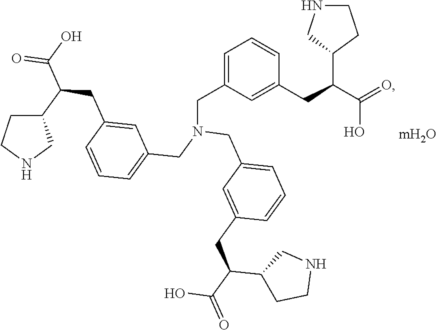 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

Formula V. Lp(a) Inhibitor Compound, (2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid hydrate (1 mol of Lp(a) inhibitor compound to 6.5 mol of water), muvalaplin hydrate (1 mol of muvalaplin to m mol of water), wherein m is the molar equivalent amount of water molecules per molecule of Lp(a) inhibitor molecule, and m is a number from about 0.33 to about 10, from about 0.5 to about 6.5, about 0.5 or about 6.5.

The oral composition can comprise from about 10 mg to about 240 mg, from about 30 mg to about 240 mg, from about 60 mg to about 240 mg, about 30 mg, about 60 mg, about 120 mg, or about 240 mg of the Lp(a) inhibitor compound. The oral composition can comprise from about 30% to about 50%, from about 35% to about 45%, or about 45%, by weight of the oral composition, of the Lp(a) inhibitor compound.

### Histidine

The oral composition of the present invention comprises histidine. The addition of histidine to the oral composition leads to the prevention and/or minimization of the formation of nitrosamine byproducts after the manufacture of the oral composition. Additionally, it has been surprisingly been found that the addition of histidine did not significantly impact other pharmaceutically relevant oral composition properties, such as disintegration time and/or total stability.

The oral composition can comprise from about from about 0.1% to about 5.0%, from about 0.25% to about 5.0%, 0.5% to about 5.0%, from about 0.25% to about 3.0%, from about 0.5% to about 3%, from about 1.0% to about 3.0%, from about 1.0% to about 2.0%, or about 1.5%, by weight of the oral composition, of histidine.

The oral composition can also comprise from about 1 mg to about 10 mg, from about 1.125 mg to about 9 mg, about 1.125 mg, about 2.25 mg, about 4.5 mg, or about 9 mg of histidine.

Other nitrosamine inhibitors can also be used in place of or in addition to histidine, such as ascorbic acid, sodium ascorbate, and/or a base, such as a sodium bicarbonate. These nitrosamine inhibitors can act through a redox scavenger mechanism or by raising the pH of the tablet to prevent and/or minimize the formation of a nitrosamine functional group. However, each of these possible alternatives has been shown herein to either impact disintegration time and/or oral composition stability.

### Bulking Agent

The oral composition of the present invention can also comprise a bulking agent. The bulking agent can be any compound added to tablets to increase their size and volume. The bulking agent can ensure the proper physical properties of the tablet, such as stability, hardness, and uniform drug distribution. Without bulking agents, tablets may not hold together properly or may not disintegrate and dissolve as intended, which can affect their efficacy.

Suitable bulking agents include lactose (e.g. spray-dried lactose, α-lactose, β-lactose, Tabletose®, various grades of Pharmatose®, Microtose® or Fast-FloC®), microcrystalline cellulose (various grades of Avicel®, Elcema®, Vivacel®, Ming Tai® or Solka-Floc®), other cellulose derivatives, sucrose, sorbitol, mannitol, dextrins, dextrans, maltodextrins, dextrose, fructose, kaolin, mannitol, sorbitol, sucrose, sugar, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, and/or sodium alginate.

The bulking agent can be microcrystalline cellulose. In an aspect, the microcrystalline cellulose is a low nitrite microcrystalline cellulose, such as Avicel® PH LN. In another aspect, the bulking agent is microcrystalline cellulose and the microcrystalline cellulose has no more than 200 μg/kg of nitrite content.

The oral composition can comprise from about 25% to about 75%, from about 40% to about 50%, or about 46.5%, by weight of the oral composition, of the bulking agent.

The oral composition can comprise from about 30 mg to about 300 mg, from about 34.875 mg to about 279 mg, about 34.875 mg, about 69.75 mg, about 139.5 mg, or about 279 mg of the bulking agent.

### Disintegrant

The oral composition of the present invention can also comprise a disintegrant. The disintegrant can be any compound added to a tablet formulation to break apart (disintegrate) the tablet when placed in aqueous environments, such as the stomach of an individual in need of treatment.

Suitable disintegrants include magnesium aluminum silicate (Veegum HV), alginic acid, alginates, microcrystalline cellulose, hydroxypropyl cellulose, other cellulose derivatives, croscarmellose sodium, crospovidone, polacrillin potassium, sodium starch glycolate, starch, pregelatinized starch, or carboxymethyl starch (e.g. Primogel® and Explotab®).

In an aspect, the disintegrant is croscarmellose sodium.

The oral composition can comprise from about 1% to about 15%, from about 3% to about 10%, about 3%, about 5%, or about 8%, by weight of the oral composition, of the disintegrant.

The oral composition can also include from about 3 mg to about 50 mg, from about 6 mg to about 48 mg, about 6 mg, about 11 mg, about 24 mg, or about 48 mg of the disintegrant.

### Binding Agent

The oral composition of the present invention can also comprise a binding agent. The binding agent can be any compound that are added to the tablet composition to form granules either in dry or liquid form during wet granulation or in dry form during dry granulation or directly compressed tablets.

Suitable binding agents include lactose (e.g. spray-dried lactose, α-lactose, β-lactose, Tabletose®, various grades of Pharmatose®, Microtose® or Fast-FloC®), microcrystalline cellulose (various grades of Avicel®, Elcema®, Vivacel®, Ming Tai® or Solka-Floc®), hydroxypropylcellulose, L-hydroxypropyl-cellulose (low-substituted), hydroxypropylmethylcellose (HPMC) (e.g. Methocel E, F and K, Metolose SH of Shin-Etsu, Ltd, such as, e.g., the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, ethylcellulose, sodium carboxymethylcellulose, other cellulose derivatives, sucrose, dextrins, mal-todextrins, starches or modified starches (including potato starch, maize starch and rice starch), calcium lactate, calcium carbonate, acacia, sodium alginate, agar, carrageenan, gelatin, guar gum, pectin, polyethylene glycol, and/or povidone.

In an aspect, the binding agent is hydroxypropyl cellulose.

The oral composition can comprise from about 1% to 5%, from about 2% to about 4%, or about 3%, by weight of the oral composition, of the binding agent.

The oral composition can comprise from about 1 mg to about 20 mg, from about 2.25 mg to about 18 mg, about 2.25 mg, about 4.5 mg, about 9 mg, or about 18 mg of the binding agent.

### Lubricant

The oral composition of the present invention can also comprise a lubricant. The lubricant can be any compound that is added to the formulation to decrease friction between the tablet's surface and the die wall cavity in which the tablet was formed. The lubricant can be used to reduce wear and tear of dies and punches. The lubricant can be added to the tablet composition powder directly or as a part of the manufacturing process.

Suitable lubricants include stearic acid, magnesium stearate, calcium stearate or other metallic stearate, talc, waxes, glycerides, light mineral oil, glyceryl behenate, hydrogenated vegetable oils, sodium stearyl fumarate, polyethylene glycols, alkyl sulfates, sodium benzoate, magnesium silicate, talc, and/or colloidal silica.

In an aspect, the lubricant is magnesium stearate.

The oral composition can comprise from about 0.5% to about 3% or about 1%, by weight of the oral composition, of the lubricant.

The oral composition can also comprise from about 0.5 mg to about 10 mg, from about 0.75 mg to about 6 mg, about 0.75 mg, about 1.5 mg, about 3 mg, or about 6 mg of the lubricant.

### Methods of Use of Oral Compositions

The oral compositions, as disclosed herein, can also be used for the treatment of conditions, disorders and diseases.

In a particular embodiment, the condition, disorder or disease is selected from: cardiovascular disease, elevated Lp(a) levels and ASCVD. In a further embodiment, compound 1, or a pharmaceutically acceptable salt thereof, is administered to individuals with elevated Lp(a) levels at risk for cardiovascular events. Individuals at risk for cardiovascular events or at risk for a cardiovascular event include those with coronary artery disease (CAD), those who have had a stroke, or those with peripheral artery disease or ASCVD risk equivalents (familial hypercholesterolemia or type 2 diabetes).

What is considered to be the normal range for Lp(a) levels varies depending on the ethnicity of the individual as described in European Heart Journal (2022) 43, 3925-3946. Elevated Lp(a) levels are levels which are above the normal range for that individual in view of their ethnicity. In a particular embodiment, elevated Lp(a) levels refers to Lp(a) plasma levels greater than or equal to 75 nmol/L, greater than or equal to 125 nmol/L, or greater than or equal to 175 nmol/L. In a particular embodiment, elevated Lp(a) levels refers to Lp(a) plasma levels greater than or equal to 30 mg/dL, greater than or equal to 50 mg/dL, or greater than or equal to 70 mg/dL. Both nmol/L and mg/dL are widely recognized and used measurement units for Lp(a) plasma levels.

In an embodiment, there is provided a method of treating or preventing coronary artery disease in an individual, the method comprising: orally administering to the individual in need thereof one of the oral compositions disclosed herein.

In an embodiment, there is provided a method of preventing cardiovascular death, myocardial infarction, urgent coronary revascularization, all-cause death or ischemic stroke in an individual with elevated Lp(a) levels, the method comprising: orally administering to the individual in need thereof one of the oral compositions disclosed herein.

In an embodiment, there is provided a method of treating acute coronary syndrome in an individual, the method comprising: orally administering to the individual in need thereof one of the oral compositions disclosed herein.

In an embodiment, there is provided a method of reducing cardiovascular events in an individual with ASCVD and elevated Lp(a) levels, the method comprising: orally administering to the individual in need thereof one of the oral compositions disclosed herein.

In an embodiment, there is provided a method of treating cardiovascular disease in an individual, the method comprising: orally administering to the individual in need thereof one of the oral compositions disclosed herein, wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided a method of treating an individual with elevated Lp(a) levels, the method comprising: orally administering to the individual in need thereof one of the oral compositions disclosed herein, wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided a method of treating an individual with elevated Lp(a) levels at risk for cardiovascular events, the method comprising: orally administering to the individual in need thereof one of the oral compositions disclosed herein, wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided a method of treating ASCVD in an individual, the method comprising: orally administering to the individual in need thereof one of the oral compositions disclosed herein, wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided a method of treating an individual with elevated Lp(a) levels, the method comprising the steps of:
i. measuring the Lp(a) plasma level of the individual;
ii. if the individual has an Lp(a) plasma level of greater than or equal to 175 nmol/L, orally administering to the individual one of the oral compositions disclosed herein.

In an embodiment, there is one of the oral compositions disclosed herein, for use in the treatment or prevention of coronary artery disease in an individual.

In an embodiment, there is provided one of the oral compositions disclosed herein, for use in the prevention of cardiovascular death, myocardial infarction, urgent coronary revascularization, all-cause death or ischemic stroke in an individual with elevated Lp(a) levels.

In an embodiment, there is provided one of the oral compositions disclosed herein, for use in the treatment of acute coronary syndrome in an individual.

In an embodiment, there is one of the oral compositions disclosed herein, for use in the reduction of cardiovascular events in an individual with ASCVD and elevated Lp(a) levels.

In an embodiment, there is provided one of the oral compositions disclosed herein, for use in the treatment of cardiovascular disease in an individual, wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided one of the oral compositions disclosed herein, for use in the treatment of an individual with elevated Lp(a) levels, wherein the compound is orally administered and wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided one of the oral compositions disclosed herein, for use in the treatment of an individual with elevated Lp(a) levels at risk for cardiovascular events, wherein the compound is orally administered and wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided one of the oral compositions disclosed herein, for use in the treatment of ASCVD in an individual, wherein the compound is orally administered and wherein the individual has an Lp(a) plasma level greater than or equal to 175 nmol/L prior to treatment. In a particular embodiment, the Lp(a) plasma level of the individual is less than 125 nmol/L after 12 weeks of treatment. In a further embodiment, the Lp(a) level of the individual is lowered by at least 150 nmol/L after 12 weeks of treatment.

In an embodiment, there is provided one of the oral compositions disclosed herein, for use in the treatment of an individual with elevated Lp(a) levels, wherein the treatment comprises the steps of:
i. measuring the Lp(a) plasma level of the individual;
ii. if the individual has an Lp(a) plasma level of greater than or equal to 175 nmol/L, orally administering to the individual one of the oral compositions disclosed herein.

As used herein, “about” means within a statistically meaningful range of a value or values such as, for example, a stated concentration, length, molecular weight, pH, sequence identity, time frame, temperature or volume. About can also mean±10%, or alternatively, it may mean having the same round-off at the specified value, for example, the expression “about 200° C.” is interpreted as having the same round-off as “200° C.”.

As used herein, “individual in need thereof” means a mammal, such as a human, with a condition, disease, disorder or symptom requiring treatment or therapy, including for example, those listed herein. In particular, the preferred individual to be treated is a human.

The term “pharmaceutically acceptable salt” as used herein refers a salt of a compound that is acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19. Salt formation can occur upon the addition of a pharmaceutically acceptable acid to form the acid addition salt or by the addition of a pharmaceutically acceptable base to form a base addition salt. Salts can also form simultaneously upon deprotection of a nitrogen or oxygen, i.e., removing the protecting group. Examples, reactions and conditions for salt formation are known to the skilled artisan.

As used herein, “treat,” “treating,” “to treat” and the like mean restraining, slowing, stopping or reversing the progression or severity of an existing condition, disease, disorder or symptom.

Plasma levels of Lp(a) may be determined using a commercially available immunoturbidimetric assay, for example, the assay available from Randox Laboratories Ltd (RX SERIES LP 3403). Agglutination occurs due to an antigen-antibody reaction between Lp(a) in a sample and anti-Lp(a) antibody adsorbed to latex particles. This agglutination is detected as an absorbance change at 700 nm proportional to the concentration of Lp(a) in the sample.

### EXAMPLES

### Preparation of Lp(a) Inhibitor Compounds

### Example 1—(2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid (muvalaplin)

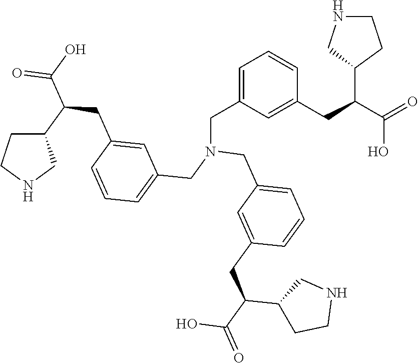 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

tert-butyl (3R)-3-[(1S)-1-[[3-[[bis[[3-[(2S)-3-tert-butoxy-2-[(3R)-1-tert-butoxycarbonylpyrrolidin-3-yl]-3-oxo-propyl]phenyl]methyl]amino]methyl]phenyl]methyl]-2-tert-butoxy-2-oxo-ethyl]pyrrolidine-1-carboxylate was prepared according to U.S. Pat. No. 11,286,249 (499.3 g, 423.3 mmol), 1,4-dioxane (1997 mL), and combined with a solution of hydrochloric acid (12 M in water, 529.1 mL, 15 equiv) in a round bottom flask. The mixture was stirred at 40° C. for 1 h and concentrated in-vacuo to remove 1,4-dioxane, which resulted in an aqueous slurry. The mixture was filtered through a propylene filter to eliminate insoluble particles. The pH of the filtrate was adjusted to 9-10 using a solution of NaOH (2M in water). The mixture was stirred at RT overnight. The resulting solid was filtered off slowly using paper filter (slow filtration, using low vacuum). The solid was washed with water and dried under vacuum at 45° C. to obtain the title compound (281 g, 88%) as a white solid. ES/MS (m/z): 711 (M+H); 1H-NMR (500 MHz, D2O) δ 7.33 (t, J=7.6 Hz, 3H), 7.27 (d, J=7.8 Hz, 3H), 7.13 (d, J=7.8 Hz, 3H), 7.09 (s, 3H), 4.20 (s, 6H), 3.54 (dd, J=7.9, 11.6 Hz, 3H), 3.39-3.34 (m, 3H), 3.23-3.17 (m, 3H), 3.02-2.98 (m, 3H), 2.84 (dd, J=4.6, 13.7 Hz, 3H), 2.76 (dd, J=10.6, 13.3 Hz, 3H), 2.60 (td, J=9.9, 4.8 Hz, 3H), 2.48 (td, J=17.3, 9.6 Hz, 3H), 2.12-2.07 (m, 3H), 1.73-1.65 (m, 3H).

### Example 2—(2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid hydrate (muvalaplin hydrate, Form A)

Example 2 was prepared by placing Example 1 into a round bottom flask with 2.75V of water and 1.75V of MeOH. A 1V KOH solution (0.24 g/g KOH/water) was added to the mixture and at 40° C. The reaction mixture was headed to 75° C. for 4 hours or until complete dissolution. The solution was filtered through a liquid material filter and placed in a new round bottom flask. The mixture was stirred at 60-90 RPM and heated to about 60° C.

A 0.25V 20% AcOH solution was dropwise added to the reaction mixture and stirred for 2 hours. A 0.72V 5% AcOH solution was dropwise added to the reaction mixture and stirred for an additional 2 hours. A 0.28V 5% AcOH solution was dropwise added to the reaction mixture and stirred for an additional 2 hours. A 1.4V 5% AcOH solution was dropwise added to the reaction mixture and stirred for an additional 2 hours. A 0.92V 5% AcOH solution was added to the reaction mixture and stirred for additional 2 hours. The pH of the mixture was adjusted, if necessary, by adding additional aliquots of 5% AcOH until the pH was from 7.5 to 7.8. The solution was cooled to 20° C. at a rate of 15° C./hr. A crystalline solid crystalized out of solution. Crystalline material was isolated and dried. Yield 87%.

Example 2 was characterized by an XRPD pattern using CuKα radiation as having diffraction peaks (2θ°) as described in Table 1 below. In an aspect, Example 2 can be characterized by an XRPD pattern using CuKα radiation as having diffraction peaks (2θ) at 14.3°±0.2° 2θ and one or more of the peaks selected from the group consisting of 4.7° and 9.5°±0.2° 2θ. In another aspect, Example 2 can be characterized by an XRPD pattern using CuKα radiation as having diffraction peaks (2θ) at 4.7°, 9.5°, and 14.3°±0.2° 2θ. In another aspect, Example 2 can be characterized by an XRPD pattern using CuKα radiation as having diffraction peaks (2θ) at 4.7°, 9.5°, 14.3°, and 19.2°±0.2° 2θ. In another aspect, Example 2 can be characterized by an XRPD pattern using CuKα radiation as having diffraction peaks (2θ) of 4.7°, 9.5°, 14.3°, 15.2°, 17.4°, and 19.2°±0.2° 2θ.

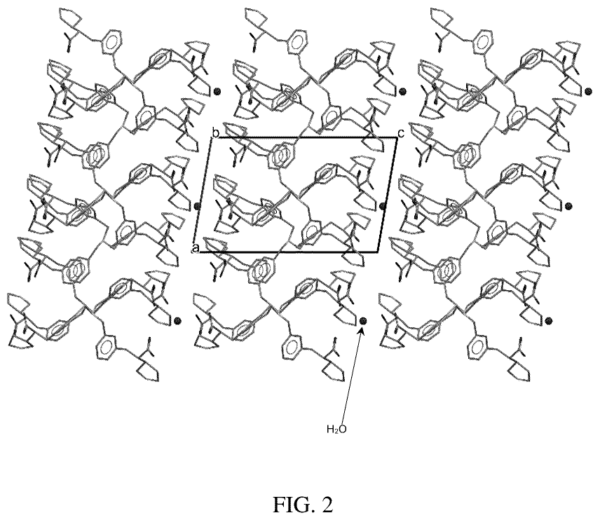

Analysis of a single crystal of Example 2 shows the material to be a hemihydrate that crystallizes in the monoclinic space group P21. Table 2 outlines the unit cell parameters and properties of Example 2. FIG. 2 shows the bonding scheme (hydrogen atoms omitted for clarity). Example 2 can be characterized by having unit cell parameters at 100 Kelvin of about a=11.25 Å, b=18.21 Å, c=18.65 Å, α=90°, β=100.19°, and γ=90°.

TABLE 1
X-ray powder diffraction peaks of Example
2, muvalaplin hydrate (Form A)

| Peak | Angle ± 0.2 (2θ°) | Relative Intensity (%) |
|---|---|---|
| 1 | 4.7 | 17.2% |
| 2 | 9.5 | 14.7% |
| 3 | 14.3 | 100.0% |
| 4 | 15.2 | 2.8% |
| 5 | 17.4 | 2.2% |
| 6 | 19.2 | 61.9% |
| 7 | 19.8 | 4.3% |

TABLE 2
Single Crystal X-Ray Diffraction of a single
crystal of Ex. 2, Muvalaplin hemihydrate

| Chemical formula | C₄₂H₅₅N₄O_{6.5} | | |
|---|---|---|---|
| Chemical formula Moiety | C₄₂H₅₄N₄O₆, 0.5(H₂O) | | |
| Formula weight | 719.91 | g/mol | |
| Temperature | 100(2) | K | |
| Wavelength | 1.54178 | Å | |
| Crystal size | 0.010 × 0.025 × 0.080 mm | | |
| Crystal habit | colorless needle | | |
| Crystal system | monoclinic | | |
| Space group | P 1 21 1 | | |
| Unit cell dimensions | a = 11.2453(11) | Å | α = 90° |
| | b = 18.2112(18) | Å | β = 100.185° |
| | c = 18.6473(17) | Å | γ = 90° |
| Volume | 3758.6(6) | Å³ | |
| Z | 4 | | |
| Z′ | 2 | | |
| Density (calculated) | 1.272 | g/cm³ | |
| Absorption coefficient | 0.690 | mm⁻¹ | |

| F(000) | 1548 | |
|---|---|---|
| Final R indices | 2445 data; I > 2σ(I) | R1 = 0.1010, |
| | | wR2 = 0.1883 |
| | all data | R1 = 0.2198, |
| | | wR2 = 0.2530 |

### Example 3—(2S)-3-[3-[[bis[[3-[(2S)-2-carboxy-2-[(3R)-pyrrolidin-3-yl]ethyl]phenyl]methyl]amino]methyl]phenyl]-2-[(3R)-pyrrolidin-3-yl]propanoic acid hydrate (muvalaplin hydrate, Form B)

Example 3 was prepared by slurrying Example 2 (muvalaplin hydrate, Form A) into a 4:1 acetone:water mixture. Form B was only stable enough to obtain a XRPD pattern if the obtained polymorph was stored at 85% RH.

Example 3 can be characterized by an XRPD pattern using CuKα radiation as having diffraction peaks (2θ) as described in Table 3 below. In an aspect, Example 3 can also be characterized by an XRPD pattern using CuKα radiation as having diffraction peaks (2θ) at 12.3°±0.2° 2θ in combination with one or more of the peaks selected from the group consisting of 8.2° and 20.6°±0.2° 2θ°. In another aspect, Example 3 can also be characterized by an XRPD pattern using CuKα radiation as having diffraction peaks (2θ) at 8.2°, 12.3°, and 20.6°±0.2° 2θ. In another aspect, Example 3 can also be characterized by an XRPD pattern using CuKα radiation as having diffraction peaks (2θ) at 8.2°, 12.3°, 16.4°, 20.6°, and 21.1°±0.2° 2θ°.

Analysis of a single crystal of Example 3 shows the material to be a 6.5 mol equivalent hydrate that crystallizes in the monoclinic space group P21. Table 4 outlines the unit cell parameters and properties of Example 3. FIG. 4 shows the bonding scheme (hydrogen atoms omitted for clarity). Example 3 can be characterized by having unit cell parameters at 100 Kelvin of about a=11.06 Å, b=18.52 Å, c=21.58 Å, α=90°, β=97.36°, and γ=90°.

TABLE 3
X-ray powder diffraction peaks of
Ex. 3, muvalaplin hydrate Form B

| Peak | Angle ± 0.2 (2θ°) | Relative Intensity (%) |
|---|---|---|
| 1 | 8.2 | 84.8% |
| 2 | 12.3 | 100.0% |
| 3 | 16.4 | 4.7% |
| 4 | 20.6 | 87.9% |
| 5 | 21.1 | 4.3% |

TABLE 4
Single Crystal X-Ray Diffraction of a single crystal of Example 3

| Chemical formula | C₄₂H₆₇N₄O_{12.50} | | |
|---|---|---|---|
| Chemical formula Moiety | C₄₂H₅₄N₄O₆, 6.5(H₂O) | | |
| Formula weight | 827.99 | g/mol | |
| Temperature | 100(2) | K | |
| Wavelength | 1.54178 | Å | |
| Crystal size | 0.015 × 0.140 × 0.140 mm | | |
| Crystal habit | clear light colorless plate | | |
| Crystal system | monoclinic | | |
| Space group | P 1 21 1 | | |
| Unit cell dimensions | a = 11.0629(7) | Å | α = 90° |
| | b = 18.5238(13) | Å | β = 97.359(4)° |
| | c = 21.5763(16) | Å | γ = 90° |
| Volume | 4385.1(5) | Å³ | |
| Z | 4 | | |
| Z′ | 2 | | |
| Density (calculated) | 1.254 | g/cm³ | |
| Absorption coefficient | 0.758 | mm⁻¹ | |

| | | |
|---|---|---|
| Final R indices | 4995 data; I > 2σ(I) | R1 = 0.2455, |
| | | wR2 = 0.4275 |
| | all data | R1 = 0.3440, |
| | | wR2 = 0.4608 |

### Method for Making Oral Compositions

Tablet compositions of TABLEs 5 and 6 were made by typical high shear wet granulation process that included security screening the powders, blending, granulation, wet milling, drying, dry milling, blending, tablet compression and coating, all techniques that are well known to a person of ordinary skill in the art. The ingredients being tested for the inhibition of nitrosamine formation, such as histidine, ascorbic acid/ascorbate, and/or sodium bicarbonate, was incorporated into the tablets by using three different methods. The three methods are described as “added dry”, “pre-treated MCC” and “aqueous solution”.

The “added dry” method was completed by adding histidine, ascorbic acid/ascorbate, and/or sodium bicarbonate with all the other excipients into the granulator at the time of the initial powder blending. This was followed by the other tablet manufacturing steps as listed above.

The “pretreated MCC” method was completed by dissolving adding histidine, ascorbic acid/ascorbate, and/or sodium bicarbonate into water and spraying the compound-to-be-tested plus water solution onto the microcrystalline cellulose. This was followed by drying. The dried, pretreated MCC, was added with all the other excipients into the granulator at the time of the initial powder blending. This was followed by the other tablet manufacturing steps as listed above.

The “aqueous solution” method was completed by dissolving adding histidine, ascorbic acid/ascorbate, and/or sodium bicarbonate into water. The other excipients are added to the granulator and blended. Then the inhibitor plus water solution is sprayed onto the powders as part of the granulation process. This was followed by the other tablet manufacturing steps as listed above.

TABLE 5
Tablet Compositions

| | | Formulation details and composition (wt %) | | | | | | | | | |
|---|---|---|---|---|---|---|---|---|---|---|---|
| Components | Role | 1 | 2 | 3 | 4 | 5 | 6 | 7 | 8 | 9 | 10 |
| Example 1 | Active | 40 | 40 | 40 | 40 | 40 | 40 | 40 | 40 | — | — |
| Example 2 | Active | — | — | — | — | | — | — | — | 41.11 | 41.11 |
| Microcrystalline cellulose¹ | Bulking agent | 48.5 | 46.5 | 46.5 | 46.5 | 46.5 | 46.5 | 45.5 | 44.5 | 44.89 | 44.89 |
| Croscarmellose sodium | Disintegrant | 8 | 8 | 8 | 8 | 8 | 8 | 8 | 8 | 8 | 8 |
| Magnesium stearate | Lubricant | 0.5 | 1.5 | 1.5 | 1.5 | 1.5 | 1.5 | 1.5 | 1.5 | 1.5 | 1.5 |
| Hydroxylpropyl cellulose | Binder | 3 | 3 | 3 | 3 | 3 | 3 | 3 | 3 | 3 | 3 |
| Histidine | | — | — | 1 | 1 | — | — | — | 1 | 1.5 | — |
| Ascorbic acid | | — | 1 | — | — | 1 | — | — | — | — | — |
| Sodium bicarbonate | | — | — | — | — | — | — | 2 | 2 | — | 2 |
| Sodium ascorbate | | — | — | — | — | — | 1 | — | — | — | — |

¹Avicel ® PH LN

TABLE 6
Additional Tablet Compositions

| | | Form. | Form. | Form. | Form. |
|---|---|---|---|---|---|
| Component | % | H-1 | H-2 | H-3 | H-4 |
| Example 2 API | 41.11 | 30.82^{a} | 61.66^{b} | 123.33^{c} | 246.66^{a} |
| Microcrystalline | 44.89 | 33.68 | 67.34 | 134.67 | 269.34 |
| cellulose | | | | | |
| CCS (intra) | 5 | 3.75 | 7.5 | 15 | 30 |
| HPC | 3 | 2.25 | 4.5 | 9 | 18 |
| Histidine (added in | 1.5 | 1.125 | 2.25 | 4.5 | 9 |
| solution) | | | | | |
| MgSt (extra) | 1.5 | 1.125 | 2.25 | 4.5 | 9 |
| CCS (extra) | 3 | 2.25 | 4.5 | 9 | 18 |
| total | 100 | 75 | 150 | 300 | 600 |

^{a}30 mg anhydrate equivalent;
^{b}60 mg anhydrate equivalent;
^{c}120 mg anhydrate equivalent; and
^{d}240 mg anhydrate equivalent

TABLE 7
Nitrosamine Content

| | | Nitrosamine Content (ppm) | | | |
|---|---|---|---|---|---|
| | | 40° C./75% RH | | | |
| Formulation | Method of Adding Nitrosamine Inhibitor | 0 mo | 1 mo | 2 mo | 3 mo |
| 1 | No Nitrosamine Inhibitor - Control | 0.191 | 2.49 | 4.82 | 3.51 |
| 2 | 1 wt % Ascorbic Acid Added Dry | 0.222 | 0.240 | 0.190 | 0.253 |
| 3 | 1 wt % Histidine Added Dry | 0.390 | 0.850 | 0.910 | 1.06 |
| 4 | 1 wt % Histidine Added Dry + Pre-Treated MCC | 0.334 | 1.00 | 1.77 | 1.61 |
| 5 | 1 wt % Ascorbic Acid Added Dry + Pre-Treated MCC | 0.181 | 0.270 | 0.180 | 0.290 |
| 6 | 1 wt % Sodium Ascorbate Added Dry | 0.245 | 0.140 | — | 0.370 |
| 7 | 2 wt % Sodium Bicarbonate Added Dry | 0.312 | 0.190 | 0.110 | 0.173 |
| 8 | 2 wt % Sodium Bicarbonate Added Dry + 1 wt % | 0.217 | 0.900 | 2.33 | 1.67 |
| | Histidine Added Dry | | | | |
| 9 | 1.5 wt % Histidine Added As Aqueous Solution | <LOQ* | <LOQ* | — | — |

*LOQ is 0.5 ppm.

TABLE 8
Disintegration Time

| | | Disintegration Time (min:sec) | |
|---|---|---|---|
| | | 40° C./75% RH | |
| Formulation | Method of Adding Nitrosamine Inhibitor | 0 mo | 1 mo |
| 1 | No Nitrosamine Inhibitor - Control | 2.84 | 4.97 |
| 2 | 1 wt % Ascorbic Acid Added Dry | 5.51 | 15.27 |
| 3 | 1 wt % Histidine Added Dry | 5.82 | 4.49 |
| 4 | 1 wt % Histidine Added Dry + Pre-Treated MCC | 6.26 | 19.26 |
| 5 | 1 wt % Ascorbic Acid Added Dry + Pre-Treated MCC | 4.48 | 15.71 |
| 6 | 1 wt % Sodium Ascorbate Added Dry | 4.87 | 12.83 |
| 7 | 2 wt % Sodium Bicarbonate Added Dry | 6.78 | 34.44 |
| 8 | 2 wt % Sodium Bicarbonate Added Dry + 1 wt % | 6.94 | 36.52 |
| | Histidine Added Dry | | |

TABLE 9
Related substances

| | | Total Related Substances | | | |
|---|---|---|---|---|---|
| | Method of Adding | (A %) 40° C./75% RH | | | |
| Formulation | Nitrosamine Inhibitor | 0 mo | 1 mo | 2 mo | 3 mo |
| 1 | No Nitrosamine | 0.70 | 0.77 | 0.66 | 0.76 |
| | Inhibitor - Control | | | | |
| 2 | 1 wt % Ascorbic Acid | 1.33 | 1.23 | 1.24 | 1.55 |
| | Added Dry | | | | |
| 3 | 1 wt % Histidine Added | 0.71 | 0.79 | 0.65 | 0.75 |
| | Dry | | | | |
| 4 | 1 wt % Histidine Added | 0.71 | 0.79 | 0.61 | — |
| | Dry + Pre-Treated MCC | | | | |
| 5 | 1 wt % Ascorbic Acid | 0.75 | 1.22 | 1.16 | — |
| | Added Dry + Pre- | | | | |
| | Treated MCC | | | | |
| 6 | 1 wt % Sodium | 0.79 | 0.92 | — | — |
| | Ascorbate Added Dry | | | | |
| 7 | 2 wt % Sodium | 0.76 | 1.09 | 1.07 | 1.64 |
| | Bicarbonate Added Dry | | | | |
| 8 | 2 wt % Sodium | 0.72 | 0.98 | 1.13 | — |
| | Bicarbonate Added Dry + | | | | |
| | 1 wt % Histidine | | | | |
| | Added Dry | | | | |

TABLEs 5 and 6 shows oral compositions of the present disclosures. TABLE 7 shows how well the oral composition of TABLE 5 prevented the formation of nitrosamine after storage under the accelerated conditions of 40° C. and 75% RH. Formulation 1, which contained no tested nitrosamine inhibitor, had an initial nitrosamine content of 0.191 ppm and 3.51 ppm after 3 months of storage under accelerated conditions. Formulation 2, which contained 1 wt % ascorbic acid added dry, had an initial nitrosamine content of 0.222 ppm and 0.253 ppm after 3 months of storage under accelerated conditions. Formulation 3, which contained 1 wt % of histidine added dry, had an initial nitrosamine content of 0.390 ppm and 1.06 ppm after 3 months of storage under accelerated conditions. Formulation 6, which contained 1 wt % sodium ascorbate added dry, had an initial nitrosamine content of 0.245 ppm and 0.370 ppm after 3 months of storage under accelerated conditions. Formulation 7, which contained 2 wt % sodium bicarbonate added dry, had an initial nitrosamine content of 0.312 ppm and 0.173 ppm after 3 months of storage under accelerated conditions. Additionally, Formulation 9, which contained histidine added as an aqueous solution, had an initial nitrosamine content and a nitrosamine content after 1 months of accelerated storage conditions that was below the limit of detection. Thus, all four tested compounds (histidine, ascorbic acid, sodium ascorbate, and sodium bicarbonate, led to the inhibition of nitrosamine relative to the tested control sample (Form. 1).

TABLE 8 shows the disintegration time of the oral compositions of TABLE 5. Formulation 1 led to an initial disintegration time of 2.84 minutes and a disintegration time of 4.97 minutes after 1 month of storage under accelerated storage conditions. Formulation 2 (1 wt % ascorbic acid) led to an initial disintegration time of 5.51 minutes and a disintegration time of 15.27 minutes after 1 month of storage under accelerated storage conditions. Formulation 3 (1 wt % histidine) led to an initial disintegration time of 5.82 minutes and a disintegration time of 4.49 minutes after 1 month of storage under accelerated storage conditions. Formulation 7 (2 wt % sodium bicarbonate) led to an initial disintegration time of 6.78 minutes and a disintegration time of 34.44 minutes after 1 month of storage under accelerated storage conditions. The formulations including ascorbic acid or sodium bicarbonate led to much higher disintegration times after 1 month storage under accelerated storage conditions relative to the control (Formulation 1). Formulation 2 (1 wt % histidine) had a disintegration time that was comparable to the control formulation, which indicated that the histidine did not impact the disintegration time of the tablet.

TABLE 9 shows the total related substances (A %) of the oral compositions of TABLE 5. A higher A % value relative to the control indicated that the excipients and/or API was reacting to the tested nitrosamine inhibitor. Formulation 1 had an initial total related substances (A %) of 0.70 and a total related substances of 0.76 after 3 months under accelerated storage conditions. Formulation 2 (1 wt % ascorbic acid) had an initial total related substances (A %) of 1.33 and a total related substances of 1.55 after 3 months under accelerated storage conditions. Formulation 6 (1 wt % sodium ascorbate) had an initial total related substances (A %) of 0.79 and a total related substances of 0.92 after 1 month under accelerated storage conditions. Formulation 7 (2 wt % of sodium bicarbonate) had an initial total related substances (A %) of 0.76 and a total related substances of 1.64 after 3 months under accelerated storage conditions. The formulations containing ascorbic acid, sodium ascorbate, and sodium bicarbonate showed significant degradation of the oral compositions relative to the control.

Unexpectedly, in total, the oral compositions including histidine did not impact the physical properties of the tablet while also preventing nitrosamine formation. Oral compositions including histidine showed a similar degradation of the oral composition relative to the control of Formulation 1. For example, Formulation 3 (1 wt % of histidine) had an initial total related substances (A %) of 0.71 and a total related substances of 0.75 after 3 months under accelerated storage conditions. Additionally, Formulation 4 (1 wt % of histidine added as a pre-treatment to microcrystalline cellulose) had an initial total related substances (A %) of 0.71 and a total related substances of 0.61 after 2 months under accelerated storage conditions.

Surprisingly, in total, the addition of the histidine led to the inhibition of the formation of nitrosamine without significantly impacting other pharmaceutically appropriate properties, such as the overall tablet stability and/or the disintegration of the oral composition.

### Methods for Determining the Amount of Nitrosamine in Oral Compositions

Each of the following methods were used to determine the amount of nitrosamine in the oral compositions comprising the Lp(a) inhibitor compounds. While not wishing to being bound by theory, it is believed that each of the below methods could be used interchangeably to determine the amount of nitrosamine in the disclosed oral compositions within normal analytical variability.

### Nitrosamine Method 1—Acidic Method Lumos:

LC-MS/MS Assay Development: Assay development was performed on an Agilent 1290 UPLC system and a Fusion Lumos tribrid mass spectrometer from Thermo Fisher Scientific (San Jose, CA). A Zorbax SB-Phenyl (2.1 mm×150 mm, 1.8 μm particle size) analytical column was used for analysis. A 3 μL injection volume was used, and the autosampler needle was washed before and after all sample injections. The column temperature was maintained at 40° C. with an autosampler temperature at 5° C. The mobile phase consisted of water with 0.1% formic acid (A) and methanol (B). The gradient was run at a flow rate of 0.3 mL/min as follows: 0 min-8 min linear ramp from 5 to 50% B, 8 min-9 min linear ramp from 50%-95%, 9 min-10 min hold at 95% B, and then ending with a re-equilibration period of 3 min at 5% B making for a 13 min method. Following LC separation, the samples were analyzed using the fusion lumos tribrid mass spectrometer with the following parameters: 45 sheath gas (arbitrary units), 6 aux gas (arbitrary units), 230° C. vaporizer temperature, 275° C. ion transfer tube temperature. The experiment is a tMS² OT CID scan with an orbitrap resolution of 60 k, isolation window of m/z 1.6, RF lens (%) of 50, microscan of 1, in negative ion mode (3.3 kV). Collision induced dissociation was used for fragmentation with a collision energy of 28%.

### Nitrosamine Method 2—Acidic Method 240:

LC-MS/MS Assay Development: Assay development was performed on a Vanquish Horizon LC system and an Exploris 240 mass spectrometer from Thermo Fisher Scientific (San Jose, CA). A Zorbax SB-Phenyl (2.1 mm×150 mm, 1.8 um particle size) analytical column was used for analysis. A 3 μL injection volume was used, and the autosampler needle was washed before and after all sample injections. The column temperature was maintained at 40° C. with an autosampler temperature at 5° C. The mobile phase consisted of water with 0.1% formic acid (A) and methanol (B). The gradient was run at a flow rate of 0.3 mL/min as follows: 0 min-8 min linear ramp from 5 to 50% B, 8 min-9 min linear ramp from 50%-95%, 9 min-10 min hold at 95% B, and then ending with a re-equilibration period of 3 min at 5% B making for a 13 min method. Following LC separation, the samples were analyzed using the Exploris 240 orbitrap mass spectrometer with the following parameters: 40 sheath gas (arbitrary units), 6 aux gas (arbitrary units), 230° C. vaporizer temperature, 275° C. ion transfer tube temperature. The experiment is a tMS² scan with an orbitrap resolution of 120 k, isolation window of m/z 2, RF lens (%) of 95, microscan of 1, in negative ion mode (3.7 kV). Higher-energy collisional dissociation was used for fragmentation with a collision energy of 40%.

Nitrosamine Method 3—Basic Method 240: DOE samples

LC-MS/MS Assay Development: Assay development was performed on an Vanquish Horizon LC system and an Exploris 240 mass spectrometer from Thermo Fisher Scientific (San Jose, CA). An Acquity Premier Protein BEH C4 (2.1 mm×50 mm, 1.7 μm particle size) analytical column was used for analysis. A 3 μL injection volume was used, and the autosampler needle was washed before and after all sample injections. The column temperature was maintained at 35° C. with an autosampler temperature at 5° C. The mobile phase consisted of water with 0.01% TEA (A) and methanol (B). The gradient was run at a flow rate of 0.3 mL/min as follows: 0 min-5 min linear ramp from 5 to 40% B, 5 min-7 min linear ramp from 40%-95%, 7 min-10 min hold at 95% B, and then ending with a re-equilibration period of 3 min at 5% B making for a 13 min method. Following LC separation, the samples were analyzed using the Exploris 240 orbitrap mass spectrometer with the following parameters: 30 sheath gas (arbitrary units), 5 aux gas (arbitrary units), 1 sweep gas (arbitrary units), 150° C. vaporizer temperature, 300° C. ion transfer tube temperature. The experiment is a tMS² scan with an orbitrap resolution of 120 k, isolation window of m/z 1, RF lens (%) of 90, microscan of 5, in negative ion mode (3.2 kV). Higher-energy collisional dissociation was used for fragmentation with a collision energy of 30%.

TABLE 10
Tablet Compositions

| | | Formulation details and composition (wt %) | | | | |
|---|---|---|---|---|---|---|
| Components | Role | 11 | 12 | 13 | 14 | 15 |
| Example 2 | Active | 41.11 | 41.11 | 41.11 | 41.11 | 41.11 |
| Microcrystalline cellulose¹ | Bulking agent | 46.39 | 46.14 | 45.89 | 43.39 | 44.89 |
| Croscarmellose sodium | Disintegrant | 8 | 8 | 8 | 10 | 8 |
| Magnesium stearate | Lubricant | 1.5 | 1.5 | 1.5 | 1.5 | 1.5 |
| Hydroxylpropyl cellulose | Binder | 3 | 3 | 3 | 3 | 3 |
| Histidine | | 0 | 0.25 | 0.50 | 1 | 1.5 |

¹Avicel ® PH LN

TABLE 11
Nitrosamine Content - 35° C./65% RH

| | 11 | 12 | 13 | 14 | 15 |
|---|---|---|---|---|---|
| 0 months | <RL* | <RL* | <RL* | <RL* | <RL* |
| 1 month | <RL* | <RL* | <RL* | <RL* | <RL* |
| 3 months | <RL* | <RL* | <RL* | <RL* | <RL* |
| 6 months | <RL* | <RL* | <RL* | 1.34 ppm | <RL* |

*RL is 1.25 ppm

TABLE 12
Nitrosamine Content - 40° C./75% RH

| | 11 | 12 | 13 | 14 | 15 |
|---|---|---|---|---|---|
| 0 months | <RL* | <RL* | <RL* | <RL* | <RL* |
| 1 month | <RL* | <RL* | <RL* | <RL* | <RL* |
| 3 months | 1.30 ppm | 1.70 ppm | 1.60 ppm | 1.46 ppm | <RL* |
| 6 months | 1.63 ppm | <RL* | <RL* | 1.60 ppm | <RL* |

*RL is 1.25 ppm

TABLE 10 shows additional oral compositions of the present disclosure. TABLE 11 shows how well the oral composition of TABLE 10 prevented the formation of nitrosamine after storage under the accelerated conditions of 35° C. and 65% RH. TABLE 12 shows how well the oral composition of TABLE 10 prevented the formation of nitrosamine after storage under the accelerated conditions of at 40° C. and 75% RH. Under both conditions, histidine reduced the formation of nitrosamine.

### Additional Analytical Methods

### X-Ray Powder Diffraction (XRPD)

The XRPD patterns of crystalline solids are obtained on a Bruker D8 Endeavor X-ray powder diffractometer, equipped with a CuKα (1.5418 Å) source and a Linxeye detector, operating at 40 kV and 40 mA. The sample is scanned between 4 and 42 20°, with a step size of 0.009 20° and a scan rate of 0.5 seconds/step, and using 0.3° primary slit opening, and 3.9° PSD opening. The dry powder is packed on a quartz or silicon sample holder and a smooth surface is obtained using a glass slide. The crystal form diffraction patterns are collected at ambient temperature and relative humidity. Crystal peak positions are determined in MDI-Jade after whole pattern shifting based on an internal NIST 675 standard with peaks at 8.853 and 26.774 20°. It is well known in crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### Single Crystal X-Ray Diffraction (SCXRD)

SCXRD was carried out on a Bruker D8 VENTURE dual-source diffractometer (serial K209362) equipped with a three-circle goniometer and a Bruker PHOTON II CPAD detector, providing an average area resolution of 7.41 pixels/mm. CuKα radiation (λ=1.54178 Å) was exclusively used for all experiments. X-rays were generated by an Incoatec IμS 3.0 microfocus sealed X-ray tube equipped with mirror optics, operating at 50 kV and 1 mA. Data acquisition employed cp and (o scans, with samples mounted on a MiTeGen Nylon Loop or MicroMount using Grade A Immersion Oil, achieving a resolution of up to 0.78 Å; the resolution cutoff is highly dependent on the quality of the measured crystal. Low-temperature experiments were performed using an open flow N₂ cold stream produced by an Oxford Cryostream 800, with all low temperature measurements fixed at 100 K.

Data collection was managed with APEX5 v2023.9-2, while cell refinement and data reduction were conducted using SAINT V8.40B (Bruker AXS LLC, 2019). An empirical multi-scan absorption correction was applied using either SADABS (2016/2) or TWINABS-2012/1 for twinned data. Structure solution was achieved using SHELXT 2018/2 or XM 2013/2 (Sheldrick, 2018), followed by refinement with SHELXL-2019/2 (Sheldrick, 2019) through using ShelXle (C. B. Huebschle, 2011). Final publication materials were prepared with Mercury (CCDC, 2024).

### Disintegration

The disintegration testing was performed by following the USP-NF <701> Disintegration test for Uncoated or Plain-Coated Tablets using purified water. The reported result is the average of 3 tablets.

### Related Substances

A reverse phase HPLC method was designed to determine the related substances of Examples 1-3. It used a 4.6 mm×150 mm Zorbax column (3.5 μm) set at 30° C. A multi-step gradient was employed with 0.05% TFA in water as mobile phase A and 0.05% TFA in acetonitrile as mobile phase B; with 1.2 ml/min flow rate The UV detector was set at 215 nm.

We claim:

1. An oral composition comprising:
(a) an Lp(a) inhibitor compound of the formula: 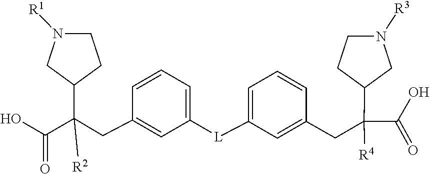
wherein
L is selected from the group consisting of —CH₂NHCH₂—, —CH₂NH—, —NH—, —S—, —S(O)—, —S(O)₂—, —O—, —OCH₂—, —OCH₂CH₂O—, —NHSO₂NH—, 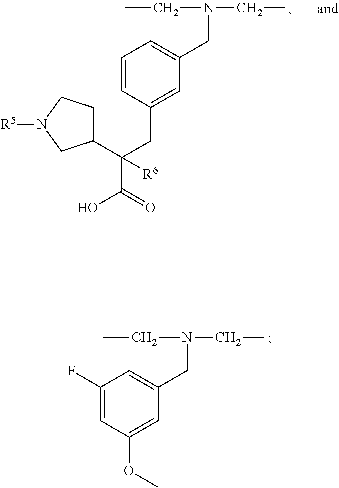
R¹, R², R³, R⁴, R⁵, and R⁶ are each independently selected from the group consisting of H and CH₃; or a pharmaceutically acceptable salt thereof; and
(b) histidine.

2. (canceled)

3. (canceled)

4. The oral composition of claim 1, wherein the Lp(a) compound is of the formula: 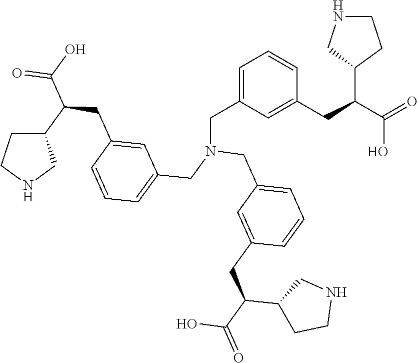 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]
or a pharmaceutically acceptable salt thereof.

5. The oral composition of claim 4, wherein the Lp(a) compound is of the formula:

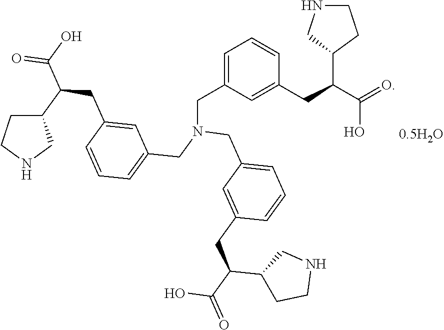 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

6. The oral composition of claim 4, wherein the Lp(a) compound is of the formula:

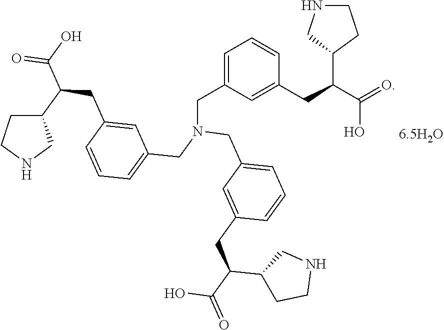 [SMILES: O=C(O)[C@@H](Cc1cccc(CN(Cc2cccc(C[C@H](C(=O)O)[C@H]3CCNC3)c2)Cc2cccc(C[C@H](C(=O)O)[C@H]3CCNC3)c2)c1)[C@H]1CCNC1]

7. The oral composition of claim 1, wherein the oral composition comprises from about 0.25 to about 5.0%, by weight of the oral composition, of histidine.

8. (canceled)

9. The oral composition of claim 1, wherein the oral composition comprises from about 1.0% to about 3.0%, by weight of the oral composition, of histidine.

10. (canceled)

11. The oral composition of claim 1, wherein the oral composition comprises about 1.5%, by weight of the oral composition, of histidine.

12. The oral composition of claim 1, wherein the oral composition comprises less than 50 ppm, less than 25 ppm, or less than 12.5 ppm of nitrosamine.

13. (canceled)

14. (canceled)

15. The oral composition of claim 1, wherein the oral composition is free of, essentially free of, or substantially free of nitrosamine.

16. The oral composition of claim 1, wherein the oral composition comprises a bulking agent, the bulking agent comprising lactose, microcrystalline cellulose, other cellulose derivatives, sucrose, sorbitol, mannitol, dextrins, dextrans, maltodextrins, dextrose, fructose, kaolin, mannitol, sorbitol, sucrose, sugar, starches or modified starches, calcium phosphate, calcium sulfate, calcium carbonate, or sodium alginate.

17-19. (canceled)

20. The oral composition of claim 16, wherein the bulking agent comprises less than 200 μg of nitrite per kg of bulking agent.

21. The oral composition of claim 1, wherein the oral composition comprises a disintegrant, the disintegrant comprising magnesium aluminum silicate, alginic acid, alginates, microcrystalline cellulose, hydroxypropyl cellulose, other cellulose derivatives, croscarmellose sodium, crospovidone, polacrillin potassium, sodium starch glycolate, starch, pregelatinized starch, or carboxymethyl starch.

22. (canceled)

23. The oral composition of claim 21, wherein the oral composition comprises from about 1% to about 15%, by weight of the oral composition, of the disintegrant.

24. (canceled)

25. The oral composition of claim 1, wherein the oral composition comprises a binding agent, the binding agent comprising lactose, microcrystalline cellulose, hydroxypropylcellulose, L-hydroxypropyl-cellulose, hydroxypropylmethylcellose (HPMC), methylcellulose polymers, hydroxyethylcellulose, ethylcellulose, sodium carboxymethylcellulose, other cellulose derivatives, sucrose, dextrins, mal-todextrins, starches or modified starches, calcium lactate, calcium carbonate, acacia, sodium alginate, agar, carrageenan, gelatin, guar gum, pectin, polyethylene glycol, or povidone.

26. (canceled)

27. The oral composition of claim 25, wherein the oral composition comprises from about 1% to about 5%, by weight of the oral composition, of the binding agent.

28-31. (canceled)

32. The oral composition of claim 1, wherein the oral composition is a tablet.

33-39. (canceled)

40. The oral composition of claim 1, wherein the oral composition comprises 30 mg, 60 mg, 120 mg, or 240 mg, anhydrate equivalent, of the Lp(a) inhibitor compound.

41. A crystalline hydrated Lp(a) inhibitor compound of the formula: 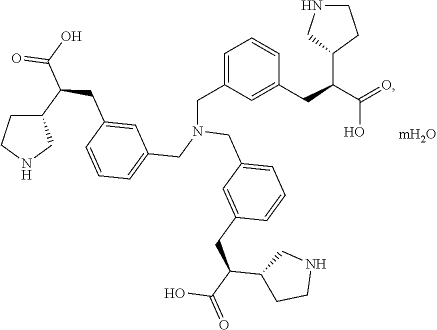
wherein m is the molar equivalent amount of water molecules per molecule of Lp(a) inhibitor molecule, and m is a number from about 0.33 to about 10.

42. The crystalline hydrated Lp(a) inhibitor compound of claim 41, wherein the compound is muvalaplin hydrate (Form A), and wherein the compound is characterized by a powder x-ray diffraction pattern using Cu Kα radiation comprising a peak at 14.3°±0.2° 2θ and one or more of the peaks selected from the group consisting of 4.7° and 9.5°±0.2° 2θ.

43. (canceled)

44. (canceled)

45. The crystalline hydrated compound of claim 42, wherein the compound is characterized by a powder x-ray diffraction pattern using Cu Kα radiation comprising peaks at 4.7°, 9.5°, 14.3°, 15.2°, 17.4°, and 19.2°±0.2° 2θ.

46. The crystalline hydrated compound of claim 45, wherein the compound is characterized by having unit cell parameters at 100 Kelvin of about a=11.25 Å, b=18.21 Å, c=18.65 Å, α=90°, β=100.19°, and γ=90°.

47. The crystalline hydrated Lp(a) inhibitor compound of claim 41, wherein the compound is muvalaplin hydrate (Form B) and wherein the compound is characterized by a powder x-ray diffraction pattern using Cu Kα radiation comprising a peak at 12.3°±0.2° 2θ in combination with one or more of the peaks selected from the group consisting of 8.2° and 20.6°±0.2° 2θ°.

48. The crystalline compound of claim 47, wherein the compound is characterized by a powder x-ray diffraction pattern using Cu Kα radiation comprising peaks at 8.2°, 12.3°, and 20.6°±0.2° 2θ.

49. The crystalline compound of claim 48, wherein the compound is characterized by a powder x-ray diffraction pattern using Cu Kα radiation comprising peaks at 8.2°, 12.3°, 16.4°, 20.6°, and 21.1°±0.2° 2θ°.

50. The crystalline compound of claim 49, wherein the compound is characterized by having unit cell parameters at 100 Kelvin of about a=11.06 Å, b=18.52 Å, c=21.58 Å, α=90°, β=97.36°, and γ=90°.

51-53. (canceled)

54. An oral composition comprising:
(a) the compound of claim 42; and
(b) the compound of claim 47.

55. The oral composition of claim 54, wherein the oral composition comprises histidine.

56-66. (canceled)
